(19) United States
(12) Patent Application Publication
(10) Pub. No.: US 2017/0105880 A1
(43) Pub. Date: Apr. 20, 2017

(54) CAST-FORMING KITS CONTAINING A LIGHTING SYSTEM AND METHODS OF MAKING AND USING THE SAME

(71) Applicants: Sabry Gabriel, Lizella, GA (US); Samy Gabriel, Lizella, GA (US)

(72) Inventors: Sabry Gabriel, Lizella, GA (US); Samy Gabriel, Lizella, GA (US)

(21) Appl. No.: 15/297,819

(22) Filed: Oct. 19, 2016

Related U.S. Application Data
(60) Provisional application No. 62/243,190, filed on Oct. 19, 2015.

(51) Int. Cl. A61F 13/04; F21V 23/04; F21V 23/02; H01M 2/10; F21V 33/00

(52) U.S. Cl. CPC A61F 13/041; H01M 2/1022; F21V 33/0068; F21V 23/023; F21V 23/04; H01M 2220/30; F21Y 2107/00

(57) ABSTRACT

Cast-forming kits and casts are disclosed. Methods of making and using cast-forming kits and casts are also disclosed.

### CROSS-REFERENCE TO RELATED APPLICATIONS

This patent application claims the benefit of priority to U.S. Provisional Patent Application Ser. No. 62/243,190 filed on Oct. 19, 2015 and entitled “CAST-FORMING KITS CONTAINING A LIGHTING SYSTEM AND METHODS OF MAKING AND USING THE SAME”, the subject matter of which is hereby incorporated by reference in its entirety.

### FIELD OF THE INVENTION

The present invention relates generally to casting materials for forming a cast on, for example, an animal's appendage. The present invention further relates to methods of making and using casting materials for forming a cast.

### BACKGROUND OF THE INVENTION

Efforts continue to further develop functional and aesthetically pleasing casting materials.

### SUMMARY OF THE INVENTION

The present invention provides new casting materials and combinations of casting materials for forming a cast. In one exemplary embodiment of the present invention, the present invention is directed to a kit of cast-forming materials for forming a cast, the kit comprising: one or more cast-forming materials; one or more lights; and a battery housing sized (i) to be transportable with a cast formed from the one or more cast-forming materials and the one or more lights, and (ii) to house one or more batteries that are suitable for providing electrical power to the one or more lights.

The present invention is also directed to casts comprising one or more lights. In one exemplary embodiment of the present invention, the cast comprises: cast material; one or more lights (i) within the cast material, (ii) along an outer surface of the cast material, or (iii) both (i) and (ii); and a battery housing attached to the cast material, the battery housing being sized (i) to be transportable with the cast, and (ii) to house one or more batteries that are suitable for providing electrical power to the one or more lights.

The present invention is further directed to methods of making casts. In one exemplary embodiment of the present invention, the method of making a cast comprises: combining one or more lights with one or more cast-forming materials so as to form a cast; and attaching a battery housing to the cast, the battery housing being sized (i) to be transportable with the cast, and (ii) to house one or more batteries that are suitable for providing electrical power to the one or more lights.

The present invention is even further directed to methods of using a cast comprising one or more lights. In one exemplary embodiment of the present invention, the method of using a cast comprises: displaying one or more lit lights (i) embedded within the cast, (ii) attached to the cast, or (iii) both (i) and (ii).

Other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a view of an exemplary kit of the present invention and the cast-forming materials contained therein;

FIG. 2 depicts a view of an exemplary cast of the present invention;

FIG. 3 depicts a view of another exemplary cast of the present invention;

FIG. 4 depicts a cross-sectional view of the exemplary cast shown in FIG. 3 along view 4-4 shown in FIG. 3;

FIG. 5 depicts a view of another exemplary cast of the present invention;

FIG. 6 depicts a cross-sectional view of a portion of the exemplary cast shown in FIG. 5 along view 6-6 shown in FIG. 5.

### DETAILED DESCRIPTION OF THE INVENTION

The present invention is directed to kits for forming casts, and casts made therefrom. The present invention is further directed to methods of making, as well as methods of using casts comprising one or more lights (i) embedded within the cast, (ii) attached to the cast, or (iii) both (i) and (ii).

One exemplary kit of the present invention is shown in FIG. 1. As shown in FIG. 1, exemplary kit 100 comprises the following exemplary kit components: cast-forming materials 20 including a settable material 21, a fibrous material 22, and a 3D printable material 23; one or more lights 30; wiring 35; a battery housing 40; one or more batteries 50 suitable for providing electrical power to the one or more lights 30; an adhesive component 60 (e.g., a double-sided adhesive tape) suitable for attaching said one or more lights 30 to an outer surface 101 of a cast 100 formed from the one or more cast-forming materials 20; one or more decorative items 70, the one or more decorative items 70 comprising (i) one or more attachable items 71 sized to be attached to an outer surface 101 of a cast 100 formed from the one or more cast-forming materials 20, and (ii) one or more markers 72 for putting indicia on an outer surface 101 of a cast 100 formed from the one or more cast-forming materials 20.

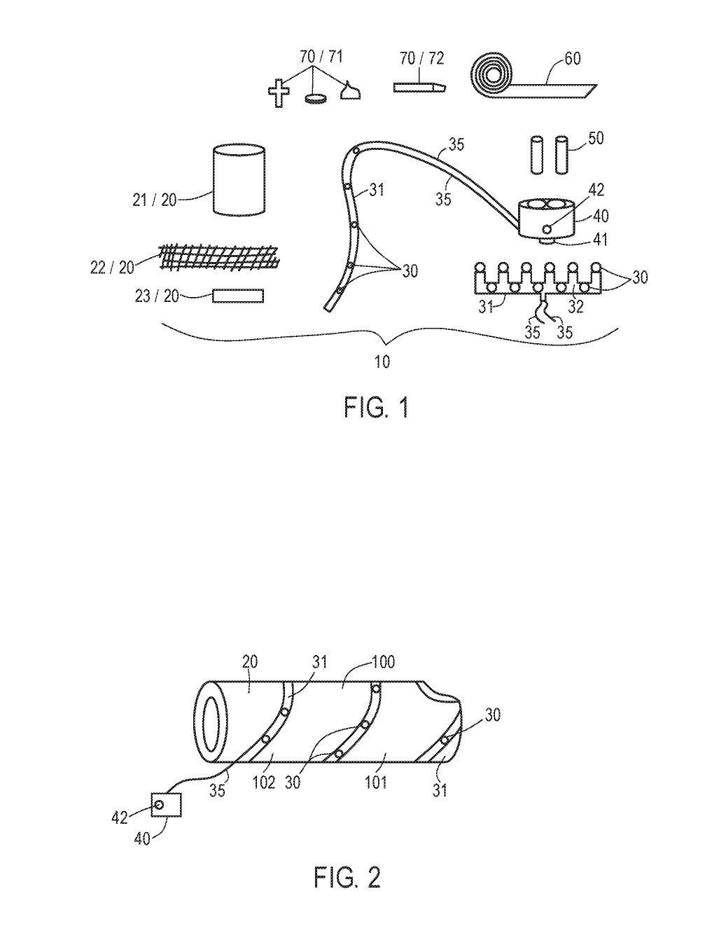

An exemplary cast 100 of the present invention is shown in FIG. 2. As shown in FIG. 2, exemplary cast 100 comprises lights 30 along an outer surface 102 of cast 100.

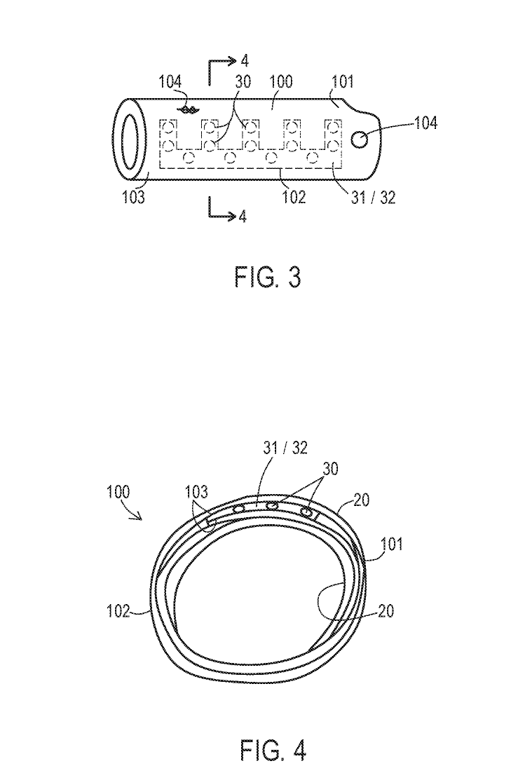

Another exemplary cast 100 of the present invention is shown in FIG. 3. As shown in FIG. 3, exemplary cast 100 comprises lights 30 within a casting material 102 (i.e., formed from the herein-described one or more cast-forming materials 20) of cast 100. FIG. 4 depicts a cross-sectional view of exemplary cast 100 shown along view 4-4 shown in FIG. 3.

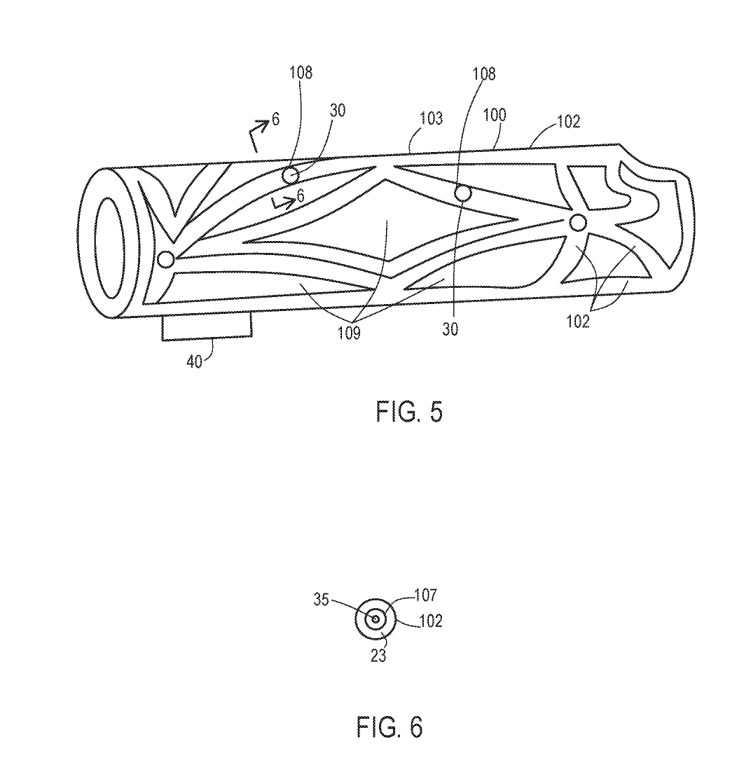

Another exemplary cast 100 of the present invention is shown in FIG. 5. As shown in FIG. 5, exemplary cast 100 comprises lights 30 within a casting material 102 (i.e., formed from the herein-described 3D printable cast-forming materials 20/23) of cast 100. In this embodiment, exemplary cast 100 comprises a web of cast material 102 with multiple apertures 109 extending through the cast 100. FIG. 6 depicts a cross-sectional view of exemplary cast 100 shown along view 6-6 shown in FIG. 5.

Further additional embodiments of the present invention are described below. It should be noted that the recitation of numerical ranges by endpoints in any of the embodiments disclosed herein includes all numbers subsumed within that range (e.g., the range about 1 to about 5 includes 1, 1.5, 2, 2.75, 3, 3.80, 4, and 5) and any range within that range (e.g., the range of about 1.5 to about 3.78 within the range of 1 to 5).

### ADDITIONAL EMBODIMENTS

Kits:

1. A kit 10 of cast-forming materials 20, said kit 10 comprising: one or more cast-forming materials 20; one or more lights 30; and a battery housing 40 sized (i) to be transportable with a cast 100 formed from the one or more cast-forming materials 20 and the one or more lights 30, and (ii) to house one or more batteries 50 that are suitable for providing electrical power to the one or more lights 30.
2. The kit 10 of embodiment 1, wherein said one or more cast-forming materials 20 comprise a settable material 21. As used herein, a “settable” material includes any material that may be used to form a cast, wherein the material hardens and/or cures via exposure to air, water, heat, light, time, or any other mechanism. Non-limiting examples include thermosettable resins (e.g., epoxies), cementitious material (e.g., plaster), etc.
3. The kit 10 of embodiment 1 or 2, wherein said one or more cast-forming materials 20 comprise a fibrous material 22. Fibrous material 22 may comprise any fiber-containing material including, but not limited to, a woven fabric/scrim/mesh, a nonwoven fabric, unidirectional fibers, a knitted fabric, strands of fibers, or any combination thereof. Further, fibrous material 22 may comprise any type of fiber including but not limited to, fiberglass fibers, polymeric fibers (e.g., nylon), elastomeric fibers, aramid fibers, ceramic fibers, cellulosic fibers (e.g., cotton), or any combination thereof.
4. The kit 10 of any one of embodiments 1 to 3, wherein said one or more cast-forming materials 20 comprise a fibrous material 22 comprising a scrim 22.
5. The kit 10 of any one of embodiments 1 to 4, wherein said one or more cast-forming materials 20 comprise a fibrous material 22 comprising a fiberglass scrim 22.
6. The kit 10 of any one of embodiments 2 to 5, wherein the fibrous material 22 is coated and/or impregnated with the settable material 21.
7. The kit 10 of any one of embodiments 2 to 6, wherein the settable material 21 comprises a polymer, a polymer-forming material, a cementitious material, or any combination thereof.
8. The kit 10 of any one of embodiments 2 to 7, wherein the settable material 21 comprises a polymer.
9. The kit 10 of any one of embodiments 2 to 7, wherein the settable material 21 comprises a polymer-forming material.
10. The kit 10 of any one of embodiments 2 to 7, wherein the settable material 21 comprises a cementitious material.
11. The kit 10 of embodiment 1, wherein said one or more cast-forming materials 20 comprise a 3D printable material 23.
12. The kit 10 of any one of embodiments 1 to 11, wherein said one or more lights 30 comprises multiple lights 30.
13. The kit 10 of any one of embodiments 1 to 12, wherein said one or more lights 30 comprises a strip 31 of multiple lights 30. Typically, strip 31 comprises multiple lights 30 within a linear configuration (i.e., a straight line).
14. The kit 10 of any one of embodiments 1 to 13, wherein said one or more lights 30 comprises an array 32 of multiple lights 30. Typically, array 32 comprises multiple lights 30 within a non-linear configuration (i.e., a pattern of lights in any configuration other than a straight line).
15. The kit 10 of any one of embodiments 1 to 14, wherein said one or more lights 30 comprises two or more lights 30 having at least two different colors of light (e.g., blue, red, white, black, orange, etc.). In some embodiments, one or more lights 30 may comprise one or more black lights.
16. The kit 10 of any one of embodiments 1 to 15, wherein said one or more lights 30 comprises one or more flashing lights 30.
17. The kit 10 of any one of embodiments 1 to 16, further comprising one or more electronic connectors 35 connecting said one or more lights 30 to (i) one another, and (ii) the battery housing 40.
18. The kit 10 of any one of embodiments 1 to 17, further comprising (i) at least one on/off switch 41 for cutting on/off said one or more lights 30, (ii) a battery charging port 42 along a surface of the battery housing 40, or (iii) both (i) and (ii).
19. The kit 10 of embodiment 18, wherein said at least one on/off switch 41 is positioned along said battery housing 40.
20. The kit 10 of any one of embodiments 1 to 19, wherein said battery housing 40 is sized to fit within a pocket of a user's shirt or pants (not shown), and said battery housing 40 is connectable to a cast 100 formed from the one or more cast-forming materials 20 and the one or more lights 30 via one or more electronic connectors 35.
21. The kit 10 of any one of embodiments 17 to 20, wherein said one or more electronic connectors 35 comprise one or more wires 35.
22. The kit 10 of any one of embodiments 1 to 21, wherein said battery housing 40 is attachable to a cast 100 formed from the one or more cast-forming materials 20 and the one or more lights 30.
23. The kit 10 of any one of embodiments 1 to 19, wherein said battery housing 40 is integrally connected to a cast 100 formed from the one or more cast-forming materials 20 and the one or more lights 30. For example, battery housing 40 may be incorporated into a cast material 102, e.g., positioned underneath one or more outer layers 103 of cast material 102, but accessible from an outer surface 101 of cast material 102.
24. The kit 10 of any one of embodiments 1 to 23, further comprising one or more batteries 50 for supplying electricity to said one or more lights 30.
25. The kit 10 of embodiment 24, wherein said one or more batteries 50 comprise one or more batteries 50 having a voltage of from about 3.0 volts (V) to about 12.0 V. It should be understood that any battery may be used in the present invention as long as the battery is transportable with the cast 100. In some embodiments, the one or more batteries 50 are rechargeable via a wired or wireless charging system (not shown). For example, a charger similar to a phone or smartphone charger (e.g., an iPhone charger) could be used to charge the one or more batteries 50 while within battery housing 40 or when separated from battery housing 40. As shown in FIGS. 1-2, battery housing 40 may comprise a battery charging port 42 along any surface of battery housing 40.
26. The kit 10 of any one of embodiments 1 to 25, further comprising an adhesive component 60 suitable for attaching said one or more lights 30 to an outer surface 101 of a cast 100 formed from the one or more cast-forming materials 20. Suitable adhesive components 60 include, but are not limited to, a double-sided tape. Alternatively, other mechanical fasteners may be used to attach one or more lights 30 to outer surface 101 of cast 100, for example, hoop and loop material.
27. The kit 10 of any one of embodiments 1 to 26, further comprising one or more decorative items 70, said one or more decorative items 70 comprising (i) one or more attachable items 71 sized to be attached to an outer surface 101 of a cast 100 formed from the one or more cast-forming materials 20, (ii) one or more markers 72 for putting indicia on an outer surface 101 of a cast 100 formed from the one or more cast-forming materials 20, or (iii) both (i) and (ii). In some embodiments, the one or more decorative items 70 comprise one or more items that glow in the presence of a black light.

Casts:

28. A cast 100 formed from the kit 10 of any one of embodiments 1 to 27.
29. A cast 100, said cast 100 comprising: cast material 102; one or more lights 30 (i) within the cast material 102, (ii) along an outer surface 101 of the cast material 102, or (iii) both (i) and (ii); and a battery housing 40 attached to the cast material 102, the battery housing 40 being sized (i) to be transportable with the cast 100, and (ii) to house one or more batteries 50 that are suitable for providing electrical power to the one or more lights 30.
30. The cast 100 of embodiment 29, wherein said cast material 102 is formed from a settable material 21.
31. The cast 100 of embodiment 29 or 30, wherein said cast material 102 comprises a fibrous material 22.
32. The cast 100 of any one of embodiments 29 to 31, wherein said cast material 102 comprises a fibrous material 22 comprising a scrim 22.
33. The cast 100 of any one of embodiments 29 to 32, wherein said cast material 102 comprises a fibrous material 22 comprising a fiberglass scrim 22.
34. The cast 100 of any one of embodiments 29 to 33, wherein said cast material 102 is formed from fibrous material 22 coated and/or impregnated with a settable material 21.
35. The cast 100 of any one of embodiments 30 to 34, wherein the settable material 21 comprises a polymer, a polymer-forming material, a cementitious material, or any combination thereof.
36. The cast 100 of any one of embodiments 30 to 35, wherein the settable material 21 comprises a polymer.
37. The cast 100 of any one of embodiments 30 to 35, wherein the settable material 21 comprises a polymer-forming material.
38. The cast 100 of any one of embodiments 30 to 35, wherein the settable material 21 comprises a cementitious material.
39. The cast 100 of embodiment 29, wherein said cast material 102 comprises a 3D printed material 23.
40. The cast 100 of any one of embodiments 28 to 39, wherein said one or more lights 30 comprises multiple lights 30.
41. The cast 100 of any one of embodiments 28 to 40, wherein said one or more lights 30 comprises a strip 31 of multiple lights 30.
42. The cast 100 of any one of embodiments 28 to 41, wherein said one or more lights 30 comprises an array 32 of multiple lights 30.
43. The cast 100 of any one of embodiments 28 to 42, wherein said one or more lights 30 comprises two or more lights 30 having at least two different colors of light.
44. The cast 100 of any one of embodiments 28 to 43, wherein said one or more lights 30 comprises one or more flashing lights 30.
45. The cast 100 of any one of embodiments 28 to 44, further comprising one or more electronic connectors 35 connecting said one or more lights 30 to (i) one another, and (ii) the battery housing 40.
46. The cast 100 of any one of embodiments 28 to 45, further comprising at least one on/off switch 41 for cutting on/off said one or more lights 30.
47. The cast 100 of embodiment 46, wherein said at least one on/off switch 41 is positioned along said battery housing 40.
48. The cast 100 of any one of embodiments 28 to 47, wherein said battery housing 40 is sized to fit within a pocket of a user's shirt or pants (not shown), and said battery housing 40 is connectable to said cast 100 via one or more electronic connectors 35.
49. The cast 100 of any one of embodiments 45 to 48, wherein said one or more electronic connectors 35 comprise one or more wires 35.
50. The cast 100 of any one of embodiments 29 to 49, wherein said battery housing 40 is attached to said cast material 102.
51. The cast 100 of any one of embodiments 29 to 50, wherein said battery housing 40 is integrally connected to said cast material 102.
52. The cast 100 of any one of embodiments 28 to 51, further comprising one or more batteries 50 for supplying electricity to said one or more lights 30.
53. The cast 100 of embodiment 52, wherein said one or more batteries 50 comprise one or more batteries 50 having a voltage of from about 3.0 volts (V) to about 12.0 V.
54. The cast 100 of embodiment 52 or 53, wherein said one or more batteries 50 are positioned within said battery housing 40.
55. The cast 100 of any one of embodiments 29 to 54, further comprising an adhesive component 60 suitable for attaching said one or more lights 30 to an outer surface 101 of said cast material 102. See, for example, exemplary cast 100 shown in FIG. 2.
56. The cast 100 of any one of embodiments 29 to 55, wherein at least a portion of said one or more lights 30 is positioned within said cast material 102. See, for example, exemplary cast 100 shown in FIGS. 3-4.
57. The cast 100 of any one of embodiments 29 to 56, wherein at least a portion of said one or more lights 30 is positioned underneath one or more outer layers 103 of said cast material 102.
58. The cast 100 of any one of embodiments 29 to 54 and 56 to 57, wherein all of said one or more lights 30 are positioned within said cast material 102.
59. The cast 100 of any one of embodiments 29 to 58, further comprising one or more decorative items 70 positioned along said cast material 102, said one or more decorative items 70 comprising (i) one or more attachable items 71 sized to be attached to an outer surface 101 of said cast material 102, (ii) indicia 104 on an outer surface 101 of said cast material 102, or (iii) both (i) and (ii).

Methods of Making a Cast:

60. A method of making a cast 100, said method comprising: using the kit 10 of any one of embodiments 1 to 27 to form a cast 100.
61. A method of making a cast 100, said method comprising: combining one or more lights 30 with one or more cast-forming materials 20 so as to form a cast 100; and attaching a battery housing 40 to the cast 100, the battery housing 40 being sized (i) to be transportable with the cast 100, and (ii) to house one or more batteries 50 that are suitable for providing electrical power to the one or more lights 30.
62. The method of embodiment 61, wherein said combining step comprises: applying the one or more cast-forming materials 20 onto an object (not shown, for example, a broken arm or leg); and incorporating the one or more lights 30 (i) onto an outer surface 101 of the one or more cast-forming materials 20, (ii) between one or more layers 103 of the one or more cast-forming materials 20, (iii) within a channel 107 or opening 108 formed by the one or more cast-forming materials 20, or (iv) any combination of (i), (ii) and (iii).
63. The method of embodiment 61 or 62, wherein said combining step comprises: incorporating the one or more lights 30 between one or more layers 103 of the one or more cast-forming materials 20.
64. The method of any one of embodiments 61 to 63, wherein said combining step comprises: incorporating the one or more lights 30 onto an outer surface 101 of the one or more cast-forming materials 20.
65. The method of any one of embodiments 61 to 64, wherein said combining step further comprises: allowing settable material 21 within the one or more cast-forming materials 20 to set.
66. The method of any one of embodiments 61 to 64, wherein said combining step further comprises: allowing a settable material 21 within the one or more cast-forming materials 20 and coated onto and/or impregnated within a fibrous material 22 to set.
67. The method of embodiment 61 or 62, wherein said combining step comprises: 3D printing a cast 100 from the one or more cast-forming materials 20.
68. The method of any one of embodiments 61, 62 and 67, wherein said combining step comprises: incorporating the one or more lights 30 within a channel 107 or opening 108 formed by the one or more cast-forming materials 20. See, for example, exemplary cast 100 shown in FIGS. 5-6.
69. The method of any one of embodiments 61 to 68, wherein said attaching step comprises: connecting the cast 100 to the battery housing 40 via one or more electrical connectors 35.
70. The method of embodiment 69, wherein the one or more electronic connectors 35 comprise one or more wires 35.
71. The method of any one of embodiments 61 to 68, wherein said combining and attaching steps occur simultaneously so as to form the battery housing 40 integrally connected to the cast 100.
72. The method of any one of embodiments 61 to 71, further comprising: providing one of more batteries 50 for supplying electricity to the one or more lights 30.
73. The method of embodiment 72, wherein the one or more batteries 50 comprise one or more batteries 50 having a voltage of from about 3.0 volts (V) to about 12.0 V.
74. The method of embodiment 71 or 72, further comprising: positioning the one of more batteries 50 within the battery housing 40.
75. The method of any one of embodiments 61 to 74, further comprising: applying an adhesive component 60 onto the one or more lights 30; and attaching the one or more lights 30 to an outer surface 101 of the cast 100 via the adhesive component 60.
76. The method of any one of embodiments 61 to 74, further comprising: applying an adhesive component 60 onto the cast 100; and attaching the one or more lights 30 to an outer surface 101 of the cast 100 via the adhesive component 60.

Methods of Using Cast-Forming Kits and/or Casts:

77. A method of using the kit 10 of any one of embodiments 1 to 27, said method comprising: forming a cast 100 with one or more lights 30 (i) embedded therein, (ii) attached thereto, or (iii) both (i) and (ii).
78. A method of using the cast 100 of any one of embodiments 28 to 59 or made by the method of any one of embodiments 60 to 76, said method comprising: displaying one or more lit lights 30 (i) embedded within the cast 100, (ii) attached to the cast 100, or (iii) both (i) and (ii).
79. The method of embodiment 78, further comprising: cutting on the one or more lit lights 100 via a switch 41.
80. The method of embodiment 78 or 79, further comprising: cutting off the one or more lit lights 30 via a switch 41.

It should be understood that although the above-described kits for forming casts, casts, and methods are described as “comprising” one or more components or steps, the above-described kits for forming casts, casts, and methods may “comprise,” “consists of,” or “consist essentially of” any of the above-described components or steps of the kits for forming casts, casts, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a kit for forming a cast, a cast, and/or a method that “comprises” a list of elements (e.g., components or steps) is not necessarily limited to only those elements (or components or steps), but may include other elements (or components or steps) not expressly listed or inherent to the kit, the cast, and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define kits for forming casts, casts, and methods that include materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that the herein-described kits for forming casts, casts, and methods may comprise, consist essentially of, or consist of any of the herein-described components and features, as shown in the figures with or without any feature(s) not shown in the figures. In other words, in some embodiments, the kits, casts, and/or methods of the present invention do not have any additional features other than those shown in the figures, and such additional features, not shown in the figures, are specifically excluded from the kits, casts, and/or methods. In other embodiments, the kits, casts, and/or methods of the present invention do have one or more additional features that are not shown in the figures.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

### Preparation of Kits and Casts

Exemplary kits and casts as shown in FIGS. 1-6 were prepared.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A kit of cast-forming materials, said kit comprising:
one or more cast-forming materials;
one or more lights; and
a battery housing sized (i) to be transportable with a cast formed from the one or more cast-forming materials and the one or more lights, and (ii) to house one or more batteries that are suitable for providing electrical power to the one or more lights.

2. The kit of claim 1, wherein said one or more cast-forming materials comprise a settable material.

3. The kit of claim 2, wherein said one or more cast-forming materials comprise a fibrous material.

4. The kit of claim 1, wherein said one or more cast-forming materials comprise a fibrous material comprising a scrim.

5. The kit of claim 3, wherein the fibrous material is coated and/or impregnated with the settable material.

6. The kit of claim 1, wherein the settable material comprises a polymer, a polymer-forming material, a cementitious material, or any combination thereof.

7. The kit of claim 1, wherein said one or more cast-forming materials comprise a 3D printable material.

8. The kit of claim 1, wherein said one or more lights comprises multiple lights.

9. The kit of claim 1, wherein said one or more lights comprises a strip of multiple lights, or an array of multiple lights.

10. The kit of claim 1, wherein said one or more lights comprises two or more lights having at least two different colors of light.

11. The kit of claim 1, wherein said one or more lights comprises one or more flashing lights.

12. The kit of claim 1, further comprising one or more electronic connectors connecting said one or more lights to (i) one another, and (ii) the battery housing.

13. The kit of claim 1, further comprising (i) at least one on/off switch for cutting on/off said one or more lights, (ii) a battery charging port along a surface of the battery housing, or (iii) both (i) and (ii).

14. The kit of claim 1, wherein said battery housing is sized to fit within a pocket of a user's shirt or pants, and said battery housing is connectable to a cast formed from the one or more cast-forming materials and the one or more lights via one or more electronic connectors.

15. The kit of claim 1, wherein said battery housing is integrally connected to a cast formed from the one or more cast-forming materials and the one or more lights.

16. The kit of claim 1, further comprising one or more batteries for supplying electricity to said one or more lights, wherein said one or more batteries comprise one or more batteries having a voltage of from about 3.0 volts (V) to about 12.0 V.

17. A cast formed from the kit of claim 1.

18. A cast, said cast comprising:
cast material;
one or more lights (i) within the cast material, (ii) along an outer surface of the cast material, or (iii) both (i) and (ii); and
a battery housing attached to the cast material, the battery housing being sized (i) to be transportable with the cast, and (ii) to house one or more batteries that are suitable for providing electrical power to the one or more lights.

19. A method of using the kit of claim 1, said method comprising:
forming a cast with one or more lights (i) embedded therein, (ii) attached thereto, or (iii) both (i) and (ii).

20. A method of using the cast of claim 17, said method comprising:
displaying one or more lit lights (i) embedded within the cast, (ii) attached to the cast, or (iii) both (i) and (ii).
